(19) United States
(12) Patent Application Publication
(10) Pub. No.: US 2022/0214347 A1
(43) Pub. Date: Jul. 7, 2022

(54) COMBINED FORMULATION KIT FOR ANALYZING PHENOTYPE AND FUNCTION OF CD1C+DENRTIC CELL SUBSET AND USE THEREOF

(71) Applicant: CAS Lamvac (Guangzhou) Biomedical Technology Co., Ltd., Guangzhou (CN)

(72) Inventors: Fang Zhou, Guangdong (CN); Xiaoping Chen, Guangdong (CN); Li Qin, Guangdong (CN); Yanli Gu, Guangdong (CN); Wenlong Xu, Guangdong (CN); Yong Lu, Guangdong (CN); Xu Chang, Guangdong (CN); Guojian Wei, Guangdong (CN); Zhien Rong, Guangdong (CN)

(21) Appl. No.: 17/607,367

(22) PCT Filed: Dec. 18, 2019
(86) PCT No.: PCT/CN2019/126389

§ 371 Date: Oct. 28, 2021

(30) Foreign Application Priority Data
Aug. 30, 2019 (CN) 201910818357.3

(51) Int. Cl. G01N 33/574; G01N 33/533

(52) U.S. Cl. CPC G01N 33/57496; G01N 33/533

(57) ABSTRACT

Disclosed are a combined formulation kit for analyzing the phenotype and function of a CD1c⁺ dendritic cell subset and the use thereof, wherein the detection objects of the kit include CD1c, CD40, IL-6 and IL-10. The kit can be used to efficiently and quickly identify the phenotype of a CD1c⁺ dendritic cell subset in peripheral blood and analyze the function thereof, thereby ensuring accuracy and reducing the economic cost produced by detecting a large number of surface antigen molecules, and the detection method is also simple to implement.

### TECHNICAL FIELD

The present application belongs to the field of biotechnology, the analysis of human peripheral blood dendritic cells by immunoassay using flow cytophotometry, and specifically relates to a combined formulation kit for the analysis of the phenotype and function of CD1c+ dendritic cell subset and use thereof.

### BACKGROUND

Flow cytometry analysis technology has been used as a major technique in immunology for both clinical and scientific research. Dendritic cells, being main regulatory cells of the immune system in the body, are one of the hotspots in immunology research. Currently, detection of dendritic cells is mainly based on flow cytometry. However, current flow cytometry assay protocols for determination of dendritic cells are diversified, lacking unified and standardized patterns. This is mainly due to rapid change in research on dendritic cells and high speed in development thereof. Several different dendritic cell subsets have been reported to be discovered. The current flow cytometry analysis protocols for dendritic cells are rough, and can no longer meet requirements for accurate analysis of different subsets of dendritic cells in clinical currently.

Flow cytometry is a device for automatic analysis and sorting of cells. It can rapidly measure, store, and display a range of important biophysically and biochemically characteristic parameters of dispersed cells suspended in liquid, and can sort out specific subsets of cells therefrom according to a pre-selected range of parameters. Most flow cytometers are instruments with a resolution of 0, that can only measure indicators such as total nucleic acid amount, total protein amount, etc. of a cell. A flow cytometry mainly consists of four components. They are the flow chamber and liquid flow system; the laser source and optical system; the photoelectric cell and detection system; and the computer and analysis system.

Flow cytometry allows simultaneous measurement of multiple parameters, with information mainly coming from specific fluorescence signals and non-fluorescence scattering signals. The measurement is performed in the measurement zone, which is the point where the irradiating laser beam and the liquid stream beam ejected from the jet hole intersect vertically. When a single cell in the center of the liquid stream passes through the measurement zone, it, upon laser irradiation, scatters light throughout the space with a stereo angle of 2π, where the wavelength of the scattered light is the same as that of the incident light. The intensity of the scattered light and its spatial distribution are closely related to the cell size, morphology, plasma membrane and internal cell structure, as these biological parameters are in turn related to the optical properties of the cell in terms of reflection and refraction of light. Cells that have not suffered any damage have a characteristic scattering for light, so that different scattered light signals can be used for the analysis and sorting of unstained live cells. The scattered light signal of fixed and stained cells is of course different from that of live cells due to the altered optical properties. The scattered light is not only related to the parameters of the cell as a scattering center, but also to abiotic factors such as the scattering angle, and the stereo angle at which the scattered light is collected.

In flow cytophotometry measurements, scattered light is commonly measured in two scattering directions: (1) forward angle (i.e., 0-angle) scatter (FSC); and (2) side scatter (SSC), also known as 90-angle scatter. In this case, the angle refers to the approximate angle between the direction of laser beam irradiation and the axial direction of the photomultiplier tube that collects the scattered light signal. In general, the intensity of the light of forward scatter is related to the size of the cell, and it increases with the cross-sectional area of the cell for homogeneous cell populations; for spherical living cells, it has been shown to be essentially linear with the cross-sectional area in a small stereo angle range; and for cells with complex shapes and orientations, it can vary greatly, and requires particular attention. Measurements of the side scatter are mainly used to obtain information about the particle properties of the fine structure inside the cell. Although the side scatter is also related to the shape and size of the cell, it is more sensitive to the refractive index of the cell membrane, cytoplasm and nuclear membrane, and also gives a sensitive reflection of the larger particles in the cytoplasm.

In practice, the instrument first measures the light scattering signal. When light scattering analysis is used in combination with a fluorescent probe, stained and unstained cells in the sample can be identified. The most effective use of light scattering measurements is to identify certain subsets from a heterogeneous population.

The fluorescence signal mainly consists of two parts: (1) autofluorescence, that is, the fluorescence emitted by the fluorescent molecules inside the cell after light irradiation without fluorescence staining; (2) characteristic fluorescence, that is, the fluorescence emitted by the fluorescent dye combined with the cell after staining by light irradiation, whose fluorescence intensity is weaker and the wavelength is different from that of the irradiated laser. The autofluorescence signal is a noise signal and in most cases interferes with the discrimination and measurement of the specific fluorescence signal. In measurements such as immunocytochemistry, it is critical to improve the signal-to-noise ratio for fluorescent antibodies that do not bind at high levels. In general, the higher the content of autofluorescence-capable molecules (e.g. riboflavin, cytochromes, etc.) in the cell composition, the stronger the autofluorescence; the higher the ratio of dead/live cells in the cultured cells, the stronger the autofluorescence; the higher the percentage of bright cells contained in the cell sample, the stronger the autofluorescence.

The main measures to reduce autofluorescence interference and improve the signal-to-noise ratio are: (1) selecting brighter fluorescent dyes as much as possible; (2) selecting suitable laser and filter optical systems; and (3) using electronic compensation circuits to compensate for the background contribution of autofluorescence.

For flow cytometry, commonly used technical indicators are fluorescence resolution, fluorescence sensitivity, applicable sample concentration, sorting purity, and analyzable measurement parameters. Flow cytometry analysis technology has become one of the most dominant techniques in the field of immunology and cell biology research.

CD1c⁺ dendritic cells are distributed in human peripheral blood and are a newly identified subset of dendritic cells in recent years. Clinical and basic studies have shown that CD1c⁺ dendritic cell subset plays an important role in the development of many diseases. For example, certain malignancies such as a lung cancer, a melanoma, a prostate cancer and a kidney cancer, dermatitis, certain viral infections such as HIV-1 infection, certain infectious diseases such as malaria infection and some autoimmune diseases such as rheumatoid arthritis. Clinical data suggests that CD1c⁺ dendritic cells show phenotypic and functional abnormalities in these diseases. Therefore, clinical data on the phenotype and function of CD1c⁺ dendritic cells can be one of the supporting indicators for clinicians to determine the development of these diseases and the effectiveness of clinical treatment. It has a very important clinical diagnostic significance.

CN105911292A discloses a kit for combinatorial analysis of CD11c⁺CD11b⁺ dendritic cell subsets and their degree of differentiation and function, comprising the following eight antibodies: CD11c, CD80, CD86, CD11b, HLA-DR, IL-12, IL-23 and IL-27. This application also provides a method for combinatorial analysis of CD11c⁺CD11b⁺ dendritic cell subsets and their degree of differentiation and function, allowing a full set of data on CD11c⁺CD11b⁺ dendritic cell subsets and their degree of differentiation and function to be detected in a single pass. However, the morphology and immune function of dendritic cells vary, and the number of antigenic molecules on their surface is large, requiring the selection of different specific detection molecules for different dendritic cell subsets. For example, it has been shown that the CD11c⁺CD11b⁺ DC subset functions quite differently from the CD1c+ DC subset and plays a role in different diseases. Therefore, the above CD11c+CD11b+ DC subset assay kits do not meet the need for studying CD1c+ DC subsets. In view of this, it is important to develop and provide an immunoassay kit for identifying the phenotype and function of CD1c+ dendritic cell subsets.

### SUMMARY

In view of shortcomings in the prior art and practical needs, the present application provides a combined formulation kit for analyzing the phenotype and function of a CD1c+ dendritic cell subset and use thereof. The combined formulation design for CD1c+ DC subsets of the present application can efficiently and quickly analyze the phenotype and function of CD1c⁺ dendritic cell subsets in peripheral blood. It ensures accuracy and reduces the economic cost caused by detecting a large number of surface antigen molecules, and the detection method is simple and easy to implement.

To achieve this, the following technical solutions are used in the present application.

In a first aspect, the present application provides a combined formulation design for analyzing the phenotype and function of a CD1c⁺ dendritic cell subset, wherein the combined formulation design comprises CD1c, CD40, IL-6 and IL-10.

In the prior art, identification of dendritic cell subsets by flow cytometry usually requires separation and extraction of peripheral blood mononuclear cells, which are complicated and cumbersome processes with a long time period. In cases where cell subsets are analyzed by detecting antigens on cell surface, a large number of antigen molecules on dendritic cell surface are usually selected for detection in order to improve the accuracy and specificity of detection. However, the detection and analysis of a large number of surface antigens takes a long time and increases the economic cost of the detection, which is not conducive to rapid and efficient analysis on dendritic cell subsets. In the present application, four molecules, namely CD1c, CD40, IL-6, and IL-10, are specifically selected. This formulation design can detect CD1c⁺ dendritic cell subsets with high specificity and sensitivity, laying a foundation for relevant scientific research.

In a second aspect, the present application provides a kit for analyzing the phenotype and function of a CD1c⁺ dendritic cell subset. The kit comprises an anti-CD1c antibody, an anti-CD40 antibody, an anti-IL-6 antibody and an anti-IL-10 antibody, wherein the anti-CD1c antibody, the anti-CD40 antibody, the anti-IL-6 antibody and the anti-IL-10 antibody are labeled with four different fluorochromes, respectively.

Kits currently available on the market for dendritic cell testing only provide a generalized analysis of overall dendritic cell data and do not include functional analysis. With the rapid development of scientific research, several new subsets of DCs, such as CD1c⁺DC, have been identified in human peripheral blood. These subsets have different phenotype and function, and there is a great need to list them separately for individual study. The existing analysis protocols obviously can not meet such a need. The analysis protocol on CD1c⁺DC phenotype and function of the present application targets the recently reported CD1c+ dendritic cell subset in human peripheral blood, and incorporates functionally-relevant cytokines (CD40, IL-6 and IL-10). The kit of the present application can provide a refined full set of data on the recently reported CD1c⁺DC subset in human peripheral blood and its function.

Preferably, the fluorochrome label is selected from FITC, PE-Cy7, PerCP-Cy5.5, Amcyan, APC-Cy7, or Q-Dot.

In a third aspect, the present application provides a method for identifying the phenotype and function of a CD1c⁺ dendritic cell subset, wherein the method adopts a combined formulation design as described in the first aspect or a kit as described in the second aspect for detection, wherein the method comprises the following steps:

(1) pretreatment of peripheral blood: separating dendritic cells, adding a leukocyte-stimulating factor and incubating;

(2) staining the blood cells obtained in step (1), then adding an anti-CD40 antibody and an anti-CD1c antibody that are labeled with different fluorochromes, carrying out a first incubation, staining again, and then fixing the obtained dendritic cells with a formalin solution, and carrying out a second incubation for later use;

(3) resuspending the cells obtained in step (2) in a cell-penetrating solution, centrifuging and discarding the supernatant, resuspending the precipitated cells in a cell-penetrating solution, adding an anti-IL-6 antibody and an anti-IL-10 antibody that are labeled with different fluorochromes, and incubating; and

(4) resuspending the incubated cells in step (3) in a cell-penetrating solution, centrifuging and discarding the supernatant, resuspending the precipitated cells in a cell-staining solution, and analyzing and detecting with a flow cytometry.

This method uses human whole blood to determine dendritic cell subsets in human peripheral blood and their function in one step, which is much simpler and easier to implement, and saves a lot of labor, material and financial resources than previous cumbersome steps of DC determination by separating peripheral blood mononuclear cells (PBMCs). Isolation of DCs with the traditional PBMC method requires a large volume of blood (usually tens of milliliters) and consumes a long time. While, we use whole blood for determination, which requires only one drop of blood (10-100 μl) from the patient to obtain the full set of information we need. It saves a lot of time in separating PBMCs, making it simple and fast to determine in one step. It is suitable for testing a large number of samples in clinical.

Preferably, the volume of peripheral blood described in step (1) is 10-100 μL, for example, 10 μL, 20 μL, 30 μL, 40 μL, 50 μL, 60 μL, 70 μL, 80 μL, 90 μL or 100 μL.

Preferably, the volume concentration of the leukocyte-stimulating factor is 0.1%-0.3%, for example, 0.1%, 0.2% or 0.3%.

Preferably, the incubation described in step (1) is carried out for 4-6 h, for example, 5.5 h or 6 h.

Preferably, the incubation described in step (1) is carried out at a temperature of 37-40° C., for example 37° C., 38° C., 39° C. or 40° C.

Preferably, the first incubation described in step (2) is carried out at room temperature for 30-60 min, for example, 30 min, 40 min, 50 min or 60 min.

Preferably, the mass fraction of the formalin solution in step (2) is 2-4%, for example, 2%, 3% or 4%.

Preferably, the second incubation described in step (2) is carried out at room temperature in the dark for 15-20 min, for example, 15 min, 16 min, 17 min, 18 min, 19 min or 20 min.

Preferably, the incubation described in step (3) is carried out at 4° C. in the dark for 12-24 h or at room temperature for 30 min.

Preferably, the analysis and detection comprise the following steps: analyzing the proportion of a dendritic cell subset having phenotype CD1c⁺ though the expression of CD1c, analyzing the differentiation and maturation status of the CD1c⁺ dendritic cell subset though the expression of CD40 molecule, and analyzing the function of the CD1c⁺ dendritic cell subset though the secretion and expression of IL-6 and IL-10.

As a preferred technical solution to the present application, the method specifically comprises the following steps:

(1) subjecting 10-100 μL of peripheral blood to anticoagulation treatment, mixing the whole peripheral blood with 1× red blood cell lysis buffer, rotating and shaking for 10 s, leaving at room temperature in the dark for 15 min, centrifuging at 350 g for 5 min, discarding the supernatant, resuspending the precipitated cells in a cell-staining solution, adding a leukocyte-stimulating factor at a volume concentration of 0.08-0.1% and incubating at 37° C. for 4-6 h;

(2) staining the blood cells obtained in step (1), then adding an anti-CD40 antibody and an anti-CD1c antibody that are labeled with different fluorochromes, incubating for 30 min at room temperature, staining again, and then fixing the obtained dendritic cells with 2% formalin solution, and incubating at room temperature in the dark for 15 min for later use;

(3) resuspending the cells obtained in step (2) in a cell-penetrating solution, centrifuging and discarding the supernatant, resuspending the precipitated cells in a cell-penetrating solution, adding an anti-IL-6 antibody and an anti-IL-10 antibody that are labeled with different fluorochromes, and incubating at 4° C. in the dark for 12 h; and

(4) resuspending the incubated cells in step (3) in a cell-penetrating solution, centrifuging and discarding the supernatant, resuspending the precipitated cells in a cell-staining solution, and analyzing and detecting by flow cytometry, analyzing the proportion of a dendritic cell subset having phenotype CD1c⁺ though the expression of CD1c, analyzing the differentiation and maturation status of the CD1c⁺ dendritic cell subset though the expression of CD40 molecule, and analyzing the function of the CD1c⁺ dendritic cell subset though the secretion and expression of IL-6 and IL-10.

Compared with the prior art, the present application has the following beneficial effects.

(1) Fast, simple and easy to implement: This method uses human whole blood to determine dendritic cell subsets in human peripheral blood and their function in one step, which is much simpler and easier to implement, and saves a lot of labor, material and financial resources than previous cumbersome steps of DC determination by separating peripheral blood mononuclear cells (PBMCs). Isolation of DCs with the traditional PBMC method requires a large volume of blood (usually tens of milliliters) and consumes a long time. While, we use whole blood for determination, which requires only one drop of blood (10-100 μl) from the patient to obtain the full set of information we need. It saves a lot of time in separating PBMCs, making it simple and fast to determine in one step. It is suitable for testing a large number of samples in clinical.

(2) Comprehensive information: the kits currently available on the market for dendritic cell testing only provide a generalized analysis of overall dendritic cell data. With the rapid development of scientific research, several new subsets of dendritic cells, such as CD1c⁺ dendritic cells, have been identified in human peripheral blood. These subsets have different phenotype and function, and there is a great need to list them separately for individual study. The existing analysis protocols obviously can not meet such a need. The analysis protocol on CD1c⁺ dendritic cell phenotype and function we designed targets the recently reported CD1c⁺dendritic cell subset in human peripheral blood, and incorporates functionally-relevant cytokines (CD40, IL-6 and IL-10), allowing us to determine the phenotype of CD1c⁺ dendritic cells and function thereof in a single step.

(3) Accuracy: Flow cytometry analysis technology is a highly sophisticated technology in the field of immunology and cell biology, which has the advantages of high sensitivity and good specificity compared with other technologies. Our analysis protocol is based on this advanced analytical technology, which makes our results more accurate and reliable.

(4) Innovativeness: The analysis protocol of dendritic cell (DC) analysis kit currently available on the market can only test data on overall DCs and do not include functional analysis. In contrast, the kit developed based on the protocol designed by us can finely provide a refined full set of data on the recently reported CD1c⁺DC subset in human peripheral blood and its function. Compared to previously developed solutions, we have combined the phenotype with function of CD1c⁺DCs for the first time for testing.

### BRIEF DESCRIPTION OF DRAWINGS

FIG. 1 shows the expression ratio of CD40 on CD1c⁺ dendritic cells in lung small cell carcinoma patients in the example;

FIG. 2 shows the expression ratio of IL-6 on CD1c⁺ dendritic cells in lung small cell carcinoma patients in the example;

FIG. 3 shows the expression ratio of IL-10 on CD1c⁺ dendritic cells in lung small cell carcinoma patients in the example;

FIG. 4 shows the expression ratio of CD40 on CD1c⁺ dendritic cells in healthy individuals in the example;

FIG. 5 shows the expression ratio of IL-6 on CD1c⁺ dendritic cells in healthy individuals in the example;

FIG. 6 shows the expression ratio of IL-10 on CD1c⁺ dendritic cells in healthy individuals in the example.

### DETAILED DESCRIPTION

In order to further illustrate the technical means adopted in the present application and effect thereof, the technical solutions of the present application are further described below by detailed description, but the present application is not limited to the scope of the examples.

Experimental Materials

Flow cytometry (BD, C6);

Anti-human CD1c, CD40, IL-6 antibodies (Biolegend) IL-10 antibody (BD).

### Example 1 Pretreatment of Peripheral Blood from Non-Small Cell Lung Cancer Patients/Healthy Individuals

The pretreatment steps are as follows:

(1) One drop (10-100 μl) of venous peripheral blood was taken from patients with non-small cell lung cancer and healthy adults, respectively, and anticoagulated.

(2) The whole peripheral blood was mixed in 2 ml 1× Red Blood Cell Lysis Buffer (Biolegend), rotated and shaken for 10 seconds and then left at room temperature for 15 min in the dark.

(3) Centrifuged in a centrifuge (350 g for 5 min), the supernatant was poured out, and the precipitated cells were suspended in 2 ml of cell-staining solution (PBS solution containing 2.5% fetal bovine serum).

(4) A leukocyte stimulating factor (BD) was added at a concentration of 0.1% and the cells were incubated at a constant temperature of 37 degrees for 6 hours.

### Example 2 Analysis of Degree of Development and Differentiation of CD1c+ Dendritic Cell Subsets in Peripheral Blood from Non-Small Cell Lung Cancer Patients/Healthy Individuals

The spare blood cells were centrifuged (350 g) for 5 minutes, the supernatant was poured out, and then the cells were suspended in 100 μl of cell-staining solution. Then 2 μl anti-human CD1c antibody and 2 μl anti-human CD40 antibody (Biolegend) were added, incubated for 30 min at room temperature, and then 2 ml of cell-staining solution was added, and centrifuged (350 g) twice, each for 5 min. After the supernatant was poured out, the cells were fixed with 2 ml of 2% formalin solution and incubated for 20 min at room temperature in the dark.

### Example 3 Functional Analysis of CD1c+ Dendritic Cell Subsets in Peripheral Blood from Non-Small Cell Lung Cancer Patients/Healthy Individuals

(1) The fixed spare cells were suspended in 2 ml of cell-penetrating solution (Biolegend) and centrifuged (350 g) for 10 min for twice.

(2) The precipitated cells were resuspended in 100 μl of cell-penetrating solution after centrifugation, added with 2 μl IL-6 antibody (Biolegend) and 2 μl IL-10 antibody (BD), and incubated for 30 min at room temperature in the dark.

(3) The incubated cells were suspended in 2 ml of cell-penetrating solution and then centrifuged (350 g) for 5 minutes for twice.

(4) Finally, the supernatant was poured out and the precipitated cells were resuspended in 0.5 ml of cell-staining solution and tested by flow cytometry analysis.

Detection and Results Analysis

1. The expression of the co-signaling stimulatory molecule CD40 in human peripheral blood CD1c⁺ dendritic cell subset was detected by flow cytometry (this data was used to assess the differentiation and maturation status of the human peripheral blood CD1c⁺ dendritic cell subset).

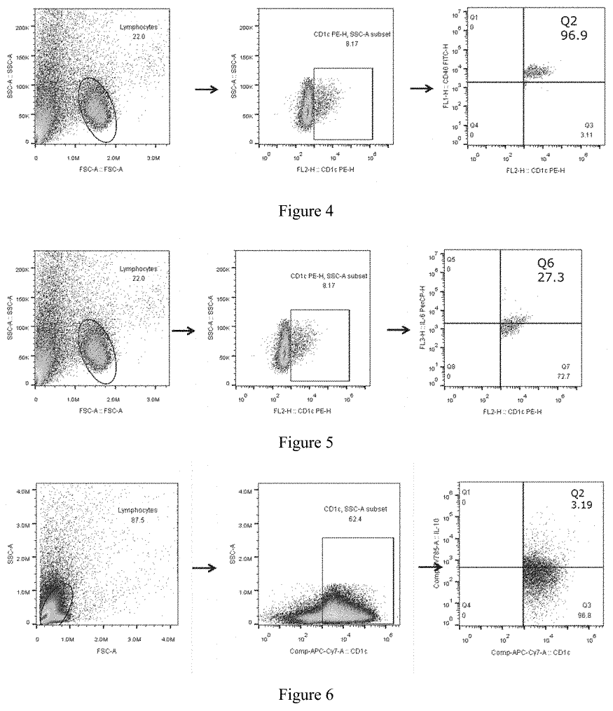

2. Functional analysis of human peripheral blood CD1c⁺ dendritic cells: the secretion and expression of cytokines IL-6 and IL-10 in CD1c⁺ dendritic cells were determined (expressed as a ratio in %). The results are shown in FIGS. 1 to 6, wherein the expression of CD40, IL-6, and IL-10 in CD1c⁺ dendritic cells in lung small cell carcinoma patients are shown in FIGS. 1 to 3, and the expression of CD40, IL-6, and IL-10 in CD1c⁺ dendritic cells in healthy individuals are shown in FIGS. 4 to 6, respectively.

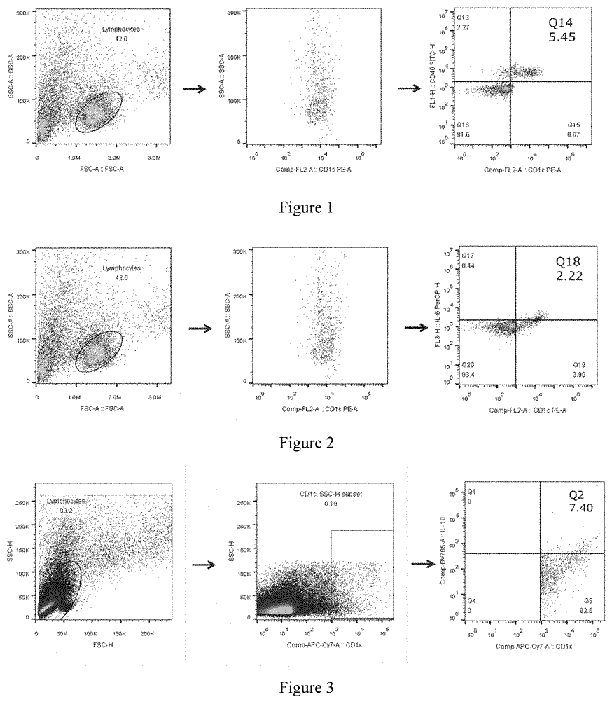

As shown in FIGS. 1 to 3, the expression ratio of CD40, IL-6, and IL-10 on CD1c⁺ dendritic cells in non-small cell lung cancer patients were 5.45%, 2.22%, and 7.4%, respectively, while the expression ratio of CD40, IL-6, and IL-10 on CD1c⁺ dendritic cells in healthy individuals were 96.9%, 27.3%, and 3.19%, respectively, demonstrating that the combined formulation design and identification method of the present application can effectively identify CD1c⁺ dendritic cell subsets in peripheral blood and analyze their differentiation and maturation as well as their function. For example, it is known that if the expression of CD40 on the surface of DCs is higher, the differentiation and maturation degree of the DCs is higher. Our results showed that the expression of CD40 on the surface of DCs in healthy individuals is significantly more than that in lung cancer patients (FIGS. 1 and 4), indicating that the differentiation and maturation degree of CD1c⁺ DCs in lung cancer patients was significantly lower than that of CD1c⁺ DCs in healthy individuals. For another example, IL-6 is a cytokine that promotes immune responses, and if DCs can secrete more IL-6, it indicates that the DCs can promote immune responses by secreting more IL-6. Our results showed that CD1c⁺ DCs in normal healthy individuals secreted significantly more IL-6 than those in lung cancer patients (FIG. 2 and FIG. 5), which indicates that the ability of CD1c⁺ DCs in lung cancer patients to enhance immune responses by secreting IL-6 is not as strong as that in healthy individuals. This is a sign of low CD1c⁺ DC-mediated immune function in lung cancer patients. In contrast, IL-10 is a cytokine that suppresses immune function, and if DCs secrete more IL-10, it indicates that the DCs have immunosuppressive efficacy and can suppress immune responses by secreting more IL-10. Our results showed that CD1c⁺ DCs in lung cancer patients precisely secreted more IL-10 than CD1c⁺ DCs in normal healthy individuals (FIG. 3 and FIG. 6). This indicates that CD1c⁺ DCs in lung cancer patients can inhibit immune function by secreting more IL-10 than CD1c⁺DCs in healthy individuals, and CD1c⁺DCs in lung cancer patients are a kind of DCs with immunosuppressive function compared with CD1c⁺DCs in healthy individuals.

In summary, the assay protocol of the present application can efficiently and rapidly compare the development and differentiation differences as well as functional differences between peripheral blood CD1c⁺DCs from patients with non-small cell lung cancer and healthy individuals. The method of the present application uses human whole blood to determine dendritic cell subsets in human peripheral blood and their function, which is simpler and easier to implement, and saves a lot of labow, material and financial resources than the traditional PBMC isolation method. The method of the present application requires only one drop of blood (10-1000 from the patient to obtain the desired full set of information. It saves a lot of time in separating PBMCs, making it simple and fast to determine in one step. It is suitable for testing a large number of samples in clinical.

1. A combined formulation design for identifying the phenotype and function of a CD1c+ dendritic cell subset, comprising CD1c, CD40, IL-6 and IL-10.

2. A method for identifying and/or preparing a product for identifying the phenotype and function of a CD1c⁺ dendritic cell subset comprising using the combined formulation design of claim 1.

3. The method according to claim 2, wherein the product comprises a kit and/or a detection reagent.

4. A kit for identifying the phenotype and function of a CD1c⁺ dendritic cell subset, comprising an anti-CD1c antibody, an anti-CD40 antibody, an anti-IL-6 antibody and an anti-IL-10 antibody, wherein the anti-CD1c antibody, the anti-CD40 antibody, the anti-IL-6 antibody and the anti-IL-10 antibodies are labeled with four different fluorochromes, respectively.

5. The kit according to claim 4, wherein the fluorochrome label is selected from FITC, PE-Cy7, PerCP-Cy5.5, Amcyan, APC-Cy7 or Q-Dot.

6. A method for identifying the phenotype and function of a CD1c⁺ dendritic cell subset, which adopts a kit of claim 4 for detection, wherein the method comprising the following steps:
(1) pretreatment of peripheral blood: separating dendritic cells, adding a leukocyte-stimulating factor and incubating;
(2) staining the blood cells obtained in step (1), then adding an anti-CD40 antibody and an anti-CD1c antibody that are labeled with different fluorochromes, carrying out a first incubation, staining again, and then fixing the obtained dendritic cells with a formalin solution, and carrying out a second incubation for later use;
(3) resuspending the cells obtained in step (2) in a cell-penetrating solution, centrifuging and discarding the supernatant, resuspending the precipitated cells in a cell-penetrating solution, adding an anti-IL-6 antibody and an anti-IL-10 antibody that are labeled with different fluorochromes, and incubating; and
(4) resuspending the incubated cells in step (3) in a cell-penetrating solution, centrifuging and discarding the supernatant, resuspending the precipitated cells in a cell-staining solution, and analyzing and detecting with a flow cytometry.

7. The method according to claim 6, wherein the volume of the peripheral blood in step (1) is 10-100 μL.

8. The method according to claim 7, wherein the volume concentration of the leukocyte-stimulating factor is 0.1%-0.3%.

9. The method according to claim 7, wherein the incubation in step (1) is carried out for 4-6 h.

10. The method according to claim 7, wherein the incubation in step (1) is carried out at a temperature of 37-40° C.

11. The method according to claim 6, wherein the first incubation in step (2) is carried out at room temperature for 30-60 min.

12. The method according to claim 11, wherein the mass fraction of the formalin solution in step (2) is 2-4%.

13. The method according to claim 6, wherein the incubation in step (3) is carried out for 12-24 h at 4° C. in the dark.

14. The method according to claim 6, wherein, the analysis and detection comprise the following steps: analyzing the proportion of a dendritic cell subset having phenotype CD1c⁺ though the expression of CD1c, analyzing the differentiation and maturation status of the CD1c⁺ dendritic cell subset though the expression of CD40 molecule, and analyzing the function of the CD1c⁺ dendritic cell subset though the secretion and expression of IL-6 and IL-10.

15. The method according to claim 6, wherein the method specifically comprising the following steps:
(1) subjecting 10-100 μL of peripheral blood to anticoagulation treatment, mixing the whole peripheral blood with 1× red blood cell lysis buffer, rotating and shaking for 10 s, leaving at room temperature in the dark for 15 min, centrifuging at 350 g for 5 min, discarding the supernatant, resuspending the precipitated cells in a cell-staining solution, adding a leukocyte-stimulating factor at a volume concentration of 0.08-0.1% and incubating at 37° C. for 4-6 h;
(2) staining the blood cells obtained in step (1), then adding an anti-CD40 antibody and an anti-CD1c antibody that are labeled with different fluorochromes, incubating for 25-35 min at room temperature, staining again, and then fixing the obtained dendritic cells with 2% formalin solution, and incubating at room temperature in the dark for 15 min for later use;
(3) resuspending the cells obtained in step (2) in a cell-penetrating solution, centrifuging and discarding the supernatant, resuspending the precipitated cells in a cell-penetrating solution, adding an anti-IL-6 antibody and an anti-IL-10 antibody that are labeled with different fluorochromes, and incubating at 4° C. in the dark for 12 h; and
(4) resuspending the incubated cells in step (3) in a cell-penetrating solution, centrifuging and discarding the supernatant, resuspending the precipitated cells in a cell-staining solution, and analyzing and detecting by flow cytometry, analyzing the proportion of a dendritic cell subset having phenotype CD1c⁺ though the expression of CD1c, analyzing the differentiation and maturation status of the CD1c⁺ dendritic cell subset though the expression of CD40 molecule, and analyzing the function of the CD1c⁺ dendritic cell subset though the secretion and expression of IL-6 and IL-10.

16. The method according to claim 11, wherein the second incubation in step (2) is carried out at room temperature in the dark for 15-20 min.
